(19) United States
(12) Patent Application Publication
(10) Pub. No.: US 2026/0364076 A1
(43) Pub. Date: Nov. 19, 2026

(54) METHODS OF TREATING CANCER WITH SOTORASIB

(71) Applicant: Amgen Inc., Thousand Oaks, CA (US)

(72) Inventors: Brett E. HOUK, Newbury Park, CA (US); Mason MCCOMB, Santa Monica, CA (US); Panli CARDONA, Burlingame, CA (US); Sandeep DUTTA, Newbury Park, CA (US)

(21) Appl. No.: 19/123,697

(22) PCT Filed: Oct. 23, 2023
(86) PCT No.: PCT/US23/35736

§ 371 Date: Apr. 23, 2025

Related U.S. Application Data
(60) Provisional application No. 63/418,695, filed on Oct. 24, 2022.

(51) Int. Cl. A61K 31/519

(52) U.S. Cl. CPC A61K 31/519

(57) ABSTRACT

The present disclosure provides methods of treating cancer in a patient comprising administering a therapeutically effective amount of sotorasib to the patient, wherein the patient has moderate or severe hepatic impairment prior to administration of the sotorasib.

### CROSS-REFERENCE TO RELATED APPLICATIONS

This application claims the benefit of priority to U.S. Provisional Patent Application No. 63/418,695, filed Oct. 24, 2022, which is hereby incorporated by reference in its entirety for all purposes.

### BACKGROUND

Sotorasib is a small molecule that specifically and irreversibly inhibits the protein product of a mutant KRAS gene with a glycine to cysteine amino acid substitution at position 12 (KRAS G12C), which encodes the KRAS^{G12C} protein. Sotorasib forms a specific covalent bond with the mutant cysteine of KRAS^{G12C}, irreversibly locking the protein in an inactive conformation that cripples oncogenic signaling (Canon, 2019). As inactivation of KRAS has been demonstrated to inhibit cell growth and/or promote apoptosis selectively in tumor cells harboring KRAS mutations (Ostrem et al., 2016; Patricelli et al., 2016; Janes et al., 2018, McDonald et al., 2017; Xie et al., 2017), sotorasib may provide a therapeutic benefit for patients with KRAS G12C-driven cancers.

### SUMMARY

Described herein are methods of treating cancer in a patient comprising administering a therapeutically effective amount of sotorasib to the patient, wherein the patient has moderate or severe hepatic impairment prior to administration of the sotorasib. Provided herein are methods comprising administering sotorasib as a free base. In various embodiments, sotorasib is administered as a pharmaceutically acceptable salt.

### DETAILED DESCRIPTION

The present disclosure is based on the discovery that patients with moderate or severe hepatic impairment can be treated with sotorasib without dose adjustment. It is understood that a reference to “a patient” or “patients” in the present disclosure refers to a subject or subjects in need of treatment, e.g., cancer treatment.

The US Food and Drug Administration's (the “FDA's”) Guidance for the Industry related to pharmacokinetics in patients with impaired hepatic function explains that “[t]he liver is involved in the clearance of many drugs through a variety of oxidative and conjugative metabolic pathways and/or through biliary excretion of unchanged drug or metabolites.” Guidance for the Industry, 2003, page 2. The FDA further notes that “[a]lterations of these excretory and metabolic activities by hepatic impairment can lead to drug accumulation . . . .” Id. Many reports in the scientific literature have shown that hepatic disease can alter the absorption and disposition of drugs (pharmacokinetics (“PK”)) as well as their efficacy and safety (pharmacodynamics, (“PD”)). Id. The FDA explains that “[e]ven though clinically useful measures of hepatic function to predict drug PK and PD are not generally available, clinical studies in patients with hepatic impairment, usually performed during drug development, can provide information that may help guide initial dosing in patients.” Id. The FDA recommends “a PK study in patients with impaired hepatic function if hepatic metabolism and/or excretion accounts for a substantial portion (>20 percent of the absorbed drug) of the elimination of a parent drug or active metabolite.” Id., page 3.

### Sotorasib

Sotorasib is a small molecule that irreversibly inhibits the KRAS^{G12C} mutant protein. Sotorasib is also referred to as AMG 510 or 6-fluoro-7-(2-fluoro-6-hydroxyphenyl)-(1M)-1-[4-methyl-2-(propan-2-yl)pyridin-3-yl]-4-[(2S)-2-methyl-4-(prop-2-enoyl)piperazin-1-yl]pyrido[2,3-d]pyrimidin-2(1H)-one and has the following structure (Formula I):

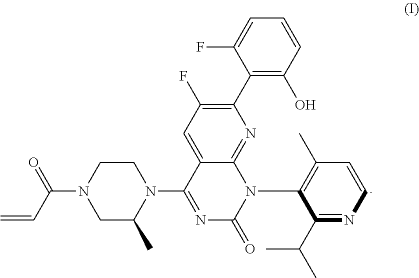

Sotorasib binds to the P2 pocket of KRAS adjacent to the mutant cysteine at position 12 and the nucleotide-binding pocket. The inhibitor contains a thiol reactive portion which covalently modifies the cysteine residue and locks KRAS^{G12C} in an jnactive, guanosine diphosphate (GDP) bound conformation. This blocks the interaction of KRAS with effectors such as rapidly accelerated fibrosarcoma (RAF), thereby preventing downstream signaling, including the phosphorylation of extracellular signal regulated kinase (ERK) (Cully and Downward, 2008; Ostrem et al., 2013; Simanshu et al., 2017). Inactivation of KRAS by RNA interference (RNAi) or small molecule inhibition has previously demonstrated an inhibition of cell growth and induction of apoptosis in tumor cell lines and xenografts harboring KRAS mutations (including the KRAS G12C mutation) (Janes et al., 2018; McDonald et al., 2017; Xie et al., 2017; Ostrem and Shokat, 2016; Patricelli et al., 2016). Studies with sotorasib have confirmed these in vitro findings and have likewise demonstrated inhibition of growth and regression of cells and tumors harboring KRAS G12C mutations (Canon et al., 2019). See also, LUMAKRAS® US Prescribing Information, Amgen Inc., Thousand Oaks, California, 91320 (revision May 2021), which is herein incorporated by reference in its entirety. In various embodiments, the methods disclosed herein comprise administering 960 mg sotorasib once daily to the patient. In various embodiments, the methods disclosed herein comprise administering 480 mg sotorasib once daily to the patient. In various embodiments, the methods disclosed herein comprise administering 240 mg sotorasib once daily to the patient.

Provided herein are methods comprising administering sotorasib as a free base. In various embodiments, sotorasib is administered as a pharmaceutically acceptable salt. For clarity the term “sotorasib” as used herein refers to the free base of sotorasib. Any method described herein referencing sotorasib can also be practiced using a pharmaceutically acceptable salt of sotorasib. In some embodiments, sotorasib can be administered as a hydrochloride, phosphate, or mesylate. In some embodiments, sotorasib can be administered as a hydrochloride. In some embodiments, sotorasib can be administered as a phosphate. In some embodiments, sotorasib can be administered as a mesylate. For clarity, if a method provided herein recites, e.g., the administration of 240 mg of sotorasib or a pharmaceutically acceptable salt thereof to a subject, the method calls for the administration 240 mg of a free base of sotorasib or the amount of a pharmaceutically acceptable salt that corresponds to the administration of 240 mg of free base of sotorasib.

The term “pharmaceutically acceptable” refers to a species or component that is generally safe, non-toxic, and neither biologically nor otherwise undesirable for use in a subject, such as a human.

The term “pharmaceutically acceptable salt” refers to a salt of a compound that possesses the desired pharmacological activity of the parent compound and that is not biologically or otherwise undesirable for its end use. Pharmaceutically acceptable salts include, for example, acid addition salts formed with inorganic acids (e.g., hydrochloric acid, hydrobromic acid, sulfuric acid, nitric acid, phosphoric acid) or formed with organic acids (e.g., acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid). Pharmaceutically acceptable salts also include, for example, salts formed when an acidic proton present in the parent compound either is replaced by a metal ion (e.g., an alkali metal ion, an alkaline earth ion, or an aluminum ion) or associates with an organic base (e.g., ethanolamine, diethanolamine, triethanolamine, N-methylglucamine, dicyclohexylamine). Additionally, the salts of the compounds described herein, can exist in either hydrated or anhydrous form or as solvates with other solvent molecules.

Sotorasib is mainly eliminated via the fecal route and mainly metabolized by the hepatic enzymes CYP3A (LUMAKRAS® US Prescribing Information, Amgen Inc., Thousand Oaks, California, 91320 (revision May 2021)), with CYP3A4 being the main metabolizing enzyme. Accordingly, patients with moderate or severe hepatic impairment would be expected to demonstrate a change in PK profile such that a certain dose of sotorasib in moderate or severely hepatically impaired patients would show, e.g., an increased exposure (“AUC”) or increased Cₘₐₓ or both compared to patients without hepatic impairment requiring a reduction of said dose to maintain an acceptable PK/PD profile. Prior studies on drug-drug interactions between sotorasib and CYP3A4 inhibitors (e.g., itraconazole) and sotorasib and rifampin (a CYP3A4 inducer) support this expectation (see, e.g., Examples 1 and 2 below). Surprisingly, however, it has been discovered that subjects with moderate or severe hepatic impairment exhibit a PK and safety profile clinically sufficiently similar to the PK and safety profile of healthy subjects (Example 3). Accordingly, the data presented in this disclosure unexpectedly shows that no dose adjustment is required for patients with moderate or severe hepatic impairment.

### Hepatic Impairment

Patients suffering from hepatic impairment includes patients diagnosed with clinical decrease in liver function, for example, due to hepatic encephalopathy, hepatitis, or cirrhosis. As used herein, a patient is diagnosed using a Child-Pugh score. While other means for measuring hepatic impairment exist (e.g., model end stage liver disease (MELD) score, Conn score), the Child-Pugh score is used herein for assessing hepatic impairment.

A Child-Pugh score (also known as the Child-Turcotte-Pugh score) is used to assess the prognosis of chronic liver disease, mainly cirrhosis, is an aggregate score of five clinical measures, billirubin, serum albumin, international normalized ratio (INR), ascites, and hepatic encephalopathy. Each marker is assigned a value from 1-3, and the total value is used to provide a score categorized as Grade A (5-6 points), Grade B (7-9 points), or Grade C (10-15 points), which can be correlated with one and two year survival rates. A patient is considered to have healthy/normal hepatic function if the patient is determined to have a Grade A on the Child-Pugh scoring scale. A patient is considered to have “moderate” hepatic impairment if the patient is determined to have a Grade B on the Child-Pugh scoring scale. A patient is considered to have “severe” hepatic impairment if the patient is determined to have Grade C on the Child-Pugh scoring scale. Methods for determination and analysis of Child-Pugh scores are well known in the art (Figg et al., 1995).

Described herein are methods of treating cancer in a patient comprising administering a therapeutically effective amount of sotorasib to the patient, wherein the patient has moderate or severe hepatic impairment prior to administration of the sotorasib. Provided herein are methods comprising administering sotorasib as a free base. In various embodiments, sotorasib is administered as a pharmaceutically acceptable salt.

The term “therapeutically effective amount” as used herein refers to that amount of a compound disclosed herein that elicits a desired biological or medical response in a cell, a tissue, a system, or a subject.

In some embodiments, the methods comprise administering a therapeutically effective amount of sotorasib to a patient that has moderate hepatic impairment (i.e., Child-Pugh Grade B) prior to administration of the sotorasib. In some embodiments, the methods comprise administering sotorasib in an amount ranging from 240 mg to 960 mg once daily to the patient with moderate hepatic impairment, In some embodiments, the methods comprise administering 960 mg sotorasib once daily to the patient with moderate hepatic impairment. In some embodiments, the methods comprise administering 480 mg sotorasib once daily to the patient with moderate hepatic impairment. In some embodiments, the methods comprise administering 240 mg sotorasib once daily to the patient with moderate hepatic impairment.

In some embodiments, the methods comprise administering a therapeutically effective amount of sotorasib to a patient that has severe hepatic impairment (i.e., Child-Pugh Grade C) prior to administration of the sotorasib. In some embodiments, the methods comprise administering sotorasib in an amount ranging from 240 mg to 960 mg once daily to the patient with severe hepatic impairment. In some embodiments, the methods comprise administering 960 mg sotorasib once daily to the patient with severe hepatic impairment. In some embodiments, the methods comprise administering 480 mg sotorasib once daily to the patient with severe hepatic impairment. In some embodiments, the methods comprise administering 240 mg sotorasib once daily to the patient with severe hepatic impairment.

### Determination of KRAS G12C Mutation in Cancer

The patient treated in the methods disclosed herein is one suffering from a cancer who has a KRAS G12C mutation. In various embodiments, the patient has a cancer that was determined to have one or more cells expressing the KRAS G12C mutant protein prior to administration as disclosed herein. The presence or absence of G12C mutation in a cancer as described herein can be determined using methods known in the art. Determining whether a tumor or cancer comprises a mutation can be undertaken, for example, by assessing the nucleotide sequence encoding the protein, by assessing the amino acid sequence of the protein, or by assessing the characteristics of a putative mutant protein or any other suitable method known in the art. The nucleotide and amino acid sequences of wild-type human KRAS (nucleotide sequence set forth in Genbank Accession No. BC010502; amino acid sequence set forth in Genbank Accession No. AGC09594) are known in the art.

Methods for detecting a mutation include, but are not limited to, polymerase chain reaction-restriction fragment length polymorphism (PCR-RFLP) assays, polymerase chain reaction-single strand conformation polymorphism (PCR-SSCP) assays, real-time PCR assays, PCR sequencing, mutant allele-specific PCR amplification (MASA) assays, direct and/or next generation-based sequencing, primer extension reactions, electrophoresis, oligonucleotide ligation assays, hybridization assays, TaqMan assays, SNP genotyping assays, high resolution melting assays and microarray analyses. In some embodiments, samples are evaluated for mutations, such as the KRAS G12C mutation, by real-time PCR. In real-time PCR, fluorescent probes specific for a certain mutation, such as the KRAS G12C mutation, are used. When a mutation is present, the probe binds and fluorescence is detected. In some embodiments, the mutation is identified using a direct sequencing method of specific regions in the gene. This technique identifies all possible mutations in the region sequenced. In some embodiments, gel electrophoresis, capillary electrophoresis, size exclusion chromatography, sequencing, and/or arrays can be used to detect the presence or absence of insertion mutations. In some embodiments, the methods include, but are not limited to, detection of a mutant using a binding agent (e.g., an antibody) specific for the mutant protein, protein electrophoresis and Western blotting, and direct peptide sequencing.

In some embodiments, multiplex PCR-based sequencing is used for mutation detection and can include a number of amplicons that provides improved sensitivity of detection of one or more genetic biomarkers. For example, multiplex PCR-based sequencing can include about 60 amplicons (e.g., 50, 51, 52, 53, 54, 55, 56, 57, 58, 59, 60, 61, 62, 63, 64, 65, 66, 67, 68, 69, or 70 amplicons). In some embodiments, multiplex PCR-based sequencing can include 61 amplicons. Amplicons produced using multiplex PCR-based sequencing can include nucleic acids having a length from about 15 bp to about 1000 bp (e.g., from about 25 bp to about 1000 bp, from about 35 bp to about 1000 bp, from about 50 bp to about 1000 bp, from about 100 bp to about 1000 bp, from about 250 bp to about 1000 bp, from about 500 bp to about 1000 bp, from about 750 bp to about 1000 bp, from about 15 bp to about 750 bp, from about 15 bp to about 500 bp, from about 15 bp to about 300 bp, from about 15 bp to about 200 bp, from about 15 bp to about 100 bp, from about 15 bp to about 80 bp, from about 15 bp to about 75 bp, from about 15 bp to about 50 bp, from about 15 bp to about 40 bp, from about 15 bp to about 30 bp, from about 15 bp to about 20 bp, from about 20 bp to about 100 bp, from about 25 bp to about 50 bp, or from about 30 bp to about 40 bp). For example, amplicons produced using multiplex PCR-based sequencing can include nucleic acids having a length of about 33 bp.

In some embodiments, the presence of one or more mutations present in a sample obtained from a patient is detected using sequencing technology (e.g., a next-generation sequencing technology). A variety of sequencing technologies are known in the art. For example, methods for detection and characterization of circulating tumor DNA in cell-free DNA can be described elsewhere (see, e.g., Haber and Velculescu, 2014). Non-limiting examples of such techniques include SafeSeqs (see, e.g., Kinde et al., 2011), OnTarget (see, e.g., Forshew et al., 2012), and TamSeq (see, e.g., Thompson et al., 2012).

In some embodiments, the presence of one or more mutations present in a sample obtained from a patient is detected using droplet digital PCR (ddPCR), a method that is known to be highly sensitive for mutation detection. In some embodiments, the presence of one or more mutations present in a sample obtained from a patient is detected using other sequencing technologies, including, but not limited to, chain-termination techniques, shotgun techniques, sequencing-by-synthesis methods, methods that utilize microfluidics, other capture technologies, or any of the other sequencing techniques known in the art that are useful for detection of small amounts of DNA in a sample (e.g., ctDNA in a cell-free DNA sample).

In some embodiments, the presence of one or more mutations present in a sample obtained from a patient is detected using array-based methods. For example, the step of detecting a genetic alteration (e.g., one or more genetic alterations) in cell-free DNA is performed using a DNA microarray. In some embodiments, a DNA microarray can detect one more of a plurality of cancer cell mutations. In some embodiments, cell-free DNA is amplified prior to detecting the genetic alteration. Non-limiting examples of array-based methods that can be used in any of the methods described herein, include: a complementary DNA (cDNA) microarray (see, e.g., Kumar et al. 2012; Laere et al. 2009; Mackay et al. 2003; DeRisi et al. 1996), an oligonucleotide microarray (see, e.g., Kim et al. 2006; Lodes et al. 2009), a bacterial artificial chromosome (BAC) clone chip (see, e.g., Chung et al. 2004; Thomas et al. 2005), a single-nucleotide polymorphism (SNP) microarray (see, e.g., Mao et al. 2007; Jasmine et al. 2012), a microarray-based comparative genomic hybridization array (array-CGH) (see, e.g., Beers and Nederlof, 2006; Pinkel et al. 2005; Michels et al. 2007), and a molecular inversion probe (MIP) assay (see, e.g., Wang et al. 2012; Lin et al. 2010). In some embodiments, the cDNA microarray is an Affymetrix microarray (see, e.g., Irizarry 2003; Dalma-Weiszhausz et al. 2006), a NimbleGen microarray (see, e.g., Wei et al. 2008; Albert et al. 2007), an Agilent microarray (see, e.g., Hughes et al. 2001), or a BeadArray array (see, e.g., Liu et al. 2017). In some embodiments, the oligonucleotide microarray is a DNA tiling array (see, e.g., Mockler and Ecker, 2005; Bertone et al. 2006). Other suitable array-based methods are known in the art.

Methods for determining whether a tumor or cancer comprises a mutation can use a variety of samples. In some embodiments, the sample is taken from a patient having a tumor or cancer. In some embodiments, the sample is afresh tumor or cancer sample. In some embodiments, the sample is a frozen tumor or cancer sample. In some embodiments, the sample is a formalin-fixed paraffin-embedded (FFPE) sample. In some embodiments, the sample is a circulating cell-free DNA and/or circulating tumor cell (CTC) sample. In some embodiments, the sample is processed to a cell lysate. In some embodiments, the sample is processed to DNA or RNA. In a certain embodiment, the sample is acquired by resection, core needle biopsy (CNB), fine needle aspiration (FNA), collection of urine, or collection of hair follicles. In some embodiments, a liquid biopsy test using whole blood or cerebral spinal fluid may be used to assess mutation status.

In various embodiments, a test approved by a regulatory authority, such as the US Food and Drug Administration (FDA), is used to determine whether the patient has a mutation, e.g., a KRAS G12C mutated cancer, or whether the tumor or tissue sample obtained from such patient contains cells with a mutation. In some embodiments, the test for a KRAS mutation used is Therascreen® KRAS RGQ PCR Kit (Qiagen). The Therascreen® KRAS RGQ PCR Kit is a real-time qualitative PCR assay for the detection of 7 somatic mutations in codons 12 and 13 of the human KRAS oncogene (G12A, G12D, G12R, G12C, G12S, G12V, and G13D) using the Rotor-Gene Q MDx 5plex HRM instrument. The kit is intended for use with DNA extracted from FFPE samples of NSCLC samples acquired by resection, CNB, or FNA. Mutation testing for STK11, KEAP1, EGFR, ALK and/or ROS1 can be conducted with commercially available tests, such as the Resolution Bioscience Resolution ctDx Lung™ assay that includes 24 genes (including those actionable in NSCLC). Tissue samples may be tested using Tempus xT 648 panel.

### KRAS G12C-Mutated Cancers

The methods described herein comprise treating a cancer with a KRAS G12C mutation in a patient. Without wishing to be bound by any particular theory, the following is noted: sotorasib is a small molecule that specifically and irreversibly inhibits KRAS^{G12C} (Hong et al., 2020). Hong et al. report that “[p]reclinical studies showed that [sotorasib]inhibited nearly all detectable phosphorylation of extracellular signal-regulated kinase (ERK), a key down-stream effector of KRAS, leading to durable complete tumor regression in mice bearing KRAS p.G12C tumors.” (id., see also Canon et al., 2019, and Lanman et al., 2020).

Sotorasib was evaluated in a Phase 1 dose escalation and expansion trial with 129 patients having histologically confirmed, locally advanced or metastatic cancer with the KRAS G12C mutation identified by local molecular testing on tumor tissues, including 59 patients with non-small cell lung cancer, 42 patients with colorectal cancer, and 28 patients with other tumor types (Hong et al., 2020, page 1208-1209). Hong et al. report a disease control rate (95% Cl) of 88.1% for non-small cell lung cancer, 73.8% for colorectal cancer and 75.0% for other tumor types (Hong et al., 2020, page 1213, Table 3). The cancer types showing either stable disease (SD) or partial response (PR) as reported by Hong et al. were non-small cell lung cancer, colorectal cancer, pancreatic cancer, appendiceal cancer, endometrial cancer, cancer of unknown primary, ampullary cancer, gastric cancer, small bowel cancer, sinonasal cancer, bile duct cancer, or melanoma (Hong et al., 2020, page 1212 (Figure A), and Supplementary Appendix (page 59 (Figure S5) and page 63 (Figure S6)).

KRAS G12C mutations occur with the alteration frequencies shown in Table 1 below (Cerami et al., 2012; Gao et al., 2013). For example, Table 1 shows that 11.6% of patients with non-small cell lung cancer have a cancer, wherein one or more cells express KRAS^{G12C} protein. Accordingly, sotorasib, which specifically and irreversibly binds to KRASG¹²c is useful for treatment of patients having a cancer, including, but not limited to the cancers listed in Table 1 below.

TABLE 1

| | Alteration |
|---|---|
| Cancer Type | Frequency |
| Non-Small Cell Lung Cancer | 11.6 |
| Small Bowel Cancer | 4.2 |
| Appendiceal Cancer | 3.6 |
| Colorectal Cancer | 3.0 |
| Cancer of Unknown Primary | 2.9 |
| Endometrial Cancer | 1.3 |
| Mixed Cancer Types | 1.2 |
| Pancreatic Cancer | 1.0 |
| Hepatobiliary Cancer | 0.7 |
| Small Cell Lung Cancer | 0.7 |
| Cervical Cancer | 0.7 |
| Germ Cell Tumor | 0.6 |
| Ovarian Cancer | 0.5 |
| Gastrointestinal Neuroendocrine | 0.4 |
| Tumor | |
| Bladder Cancer | 0.4 |
| Myelodysplastic/Myeloproliferative | 0.3 |
| Neoplasms | |
| Head and Neck Cancer | 0.3 |
| Esophagogastric Cancer | 0.2 |
| Soft Tissue Sarcoma | 0.2 |
| Mesothelioma | 0.2 |
| Thyroid Cancer | 0.1 |
| Leukemia | 0.1 |
| Melanoma | 0.1 |

In various embodiments, the cancer is a solid tumor. In various embodiments, the cancer is non-small cell lung cancer, small bowel cancer, appendiceal cancer, colorectal cancer, cancer of unknown primary, endometrial cancer, pancreatic cancer, hepatobiliary cancer, small cell lung cancer, cervical cancer, germ cell cancer, ovarian cancer, gastrointestinal neuroendocrine cancer, bladder cancer, myelodysplastic/myeloproliferative neoplasms, head and neck cancer, esophagogastric cancer, soft tissue sarcoma, mesothelioma, thyroid cancer, leukemia, melanoma, ampullary cancer, gastric cancer, sinonasal cancer, or bile duct cancer. In some embodiments, the cancer is non-small cell lung cancer, small bowel cancer, appendiceal cancer, colorectal cancer, cancer of unknown primary, endometrial cancer, pancreatic cancer, melanoma, ampullary cancer, gastric cancer, sinonasal cancer, or bile duct cancer. In various embodiments, the cancer is non-small cell lung cancer, and in some specific embodiments, metastatic or locally advanced non-small cell lung cancer. In various embodiments, the cancer is colorectal cancer. In some embodiments, the cancer is pancreatic cancer. In one embodiment, any of the above-identified cancers is a KRAS G12C-mutated cancer.

### Embodiments

1. A method of treating cancer in a patient comprising administering a therapeutically effective amount of sotorasib to the patient, wherein the patient has moderate or severe hepatic impairment prior to administration of the sotorasib.
2. The method of embodiment 1, wherein the patient has moderate hepatic impairment prior to administration of the sotorasib.
3. The method of embodiment 1, wherein the patient has severe hepatic impairment prior to administration of the sotorasib.
4. The method of embodiment 1 or embodiment 2, comprising administering 960 mg sotorasib once daily to the patient.
5. The method of embodiment 1, comprising administering 240 mg sotorasib once daily to the patient.
6. The method of any one of embodiments 1 to 5, wherein the patient has a cancer comprising a KRAS G12C mutation.
7. The method of embodiment 6, wherein the cancer is a solid tumor.
8. The method of embodiment 6 or 7, wherein the cancer is non-small cell lung cancer, small bowel cancer, appendiceal cancer, colorectal cancer, cancer of unknown primary, endometrial cancer, pancreatic cancer, hepatobiliary cancer, small cell lung cancer, cervical cancer, germ cell cancer, ovarian cancer, gastrointestinal neuroendocrine cancer, bladder cancer, myelodysplastic/myeloproliferative neoplasms, head and neck cancer, esophagogastric cancer, soft tissue sarcoma, mesothelioma, thyroid cancer, leukemia, melanoma, ampullary cancer, gastric cancer, sinonasal cancer, or bile duct cancer.
9. The method of any one of embodiments 6 to 8, wherein the cancer is non-small cell lung cancer, small bowel cancer, appendiceal cancer, colorectal cancer, cancer of unknown primary, endometrial cancer, pancreatic cancer, melanoma, ampullary cancer, gastric cancer, sinonasal cancer, or bile duct cancer.
10. The method of any one of embodiments 6 to 9, wherein the cancer is non-small cell lung cancer.
11. The method of embodiment 10, wherein the non-small cell lung cancer is locally advanced or metastatic.
12. The method of any one of embodiments 6 to 9, wherein the cancer is colorectal cancer.
13. The method of any one of embodiments 6-9, wherein the cancer is pancreatic cancer.

### Alternative Embodiments

1. A method of treating cancer in a patient comprising administering a therapeutically effective amount of sotorasib to the patient, wherein the patient had moderate or severe hepatic impairment prior to administration of the sotorasib, wherein sotorasib is a compound of Formula (I)

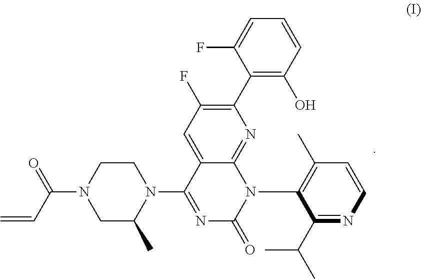

2. The method of claim 1, wherein the patient had moderate hepatic impairment prior to administration of the sotorasib.
3. The method of claim 1, wherein the patient had severe hepatic impairment prior to administration of the sotorasib.
4. The method of claim 1 or claim 2, wherein the method further comprises a step of determining whether the patient has moderate hepatic impairment.
5. The method of claim 1 or claim 3, wherein the method further comprises a step of determining whether the patient has severe hepatic impairment.
6. The method of any one of claims 1 to 5, wherein the therapeutically effective amount of sotorasib is 240 mg to 960 mg daily.
7. The method of any one of claims 1 to 6, wherein the therapeutically effective amount of sotorasib is 240 mg sotorasib daily.
8. The method of any one of claims 1 to 6, wherein the therapeutically effective amount of sotorasib is 480 mg daily.
9. The method of any one of claims 1 to 6, wherein the therapeutically effective amount of sotorasib is 960 mg daily.
10. The method of any one of claims 1 to 9, wherein the patient has a cancer comprising a KRAS G12C mutation.
11. The method of claim 10, wherein the method further comprises determining whether the patient has a cancer comprising a KRAS G12C mutation.
12. The method of any one of claims 1 to 11, wherein the cancer is a solid tumor.
13. The method of any one of claims 1 to 11, wherein the cancer is non-small cell lung cancer, small bowel cancer, appendiceal cancer, colorectal cancer, cancer of unknown primary, endometrial cancer, pancreatic cancer, hepatobiliary cancer, small cell lung cancer, cervical cancer, germ cell cancer, ovarian cancer, gastrointestinal neuroendocrine cancer, bladder cancer, myelodysplastic/myeloproliferative neoplasms, head and neck cancer, esophagogastric cancer, soft tissue sarcoma, mesothelioma, thyroid cancer, leukemia, melanoma, ampullary cancer, gastric cancer, sinonasal cancer, or bile duct cancer.
14. The method of any one of claims 1 to 11, wherein the cancer is non-small cell lung cancer, small bowel cancer, appendiceal cancer, colorectal cancer, cancer of unknown primary, endometrial cancer, pancreatic cancer, melanoma, ampullary cancer, gastric cancer, sinonasal cancer, or bile duct cancer.
15. The method of any one of claims 1 to 11, wherein the cancer is non-small cell lung cancer.
16. The method of claim 15, wherein the non-small cell lung cancer is locally advanced or metastatic.
17. The method of any one of claims 1 to 11, wherein the cancer is colorectal cancer.
18. The method of any one of claims 1 to 11, wherein the cancer is pancreatic cancer.
19. The method of any one of claims 1 to 18, where the sotorasib is administered as one or more tablets.
20. The method of any one of claims 1 to 19, wherein the sotorasib is administered orally.
21. The method of any one of claims 1 to 20, wherein the sotorasib is administered once daily.

### EXAMPLES

### Example 1—an Open-Label Study to Evaluate the Drug-Drug Interaction Effect of Itraconazole, a CYP3A4 Inhibitor, on the Pharmacokinetics of Sotorasib in Healthy Subjects

### Objectives:

This was a phase 1, single-center, open-label, fixed-sequence study to investigate the effect of coadministration of itraconazole (CYP3A4 and P-gp inhibitor) on the PK of sotorasib in healthy men and women of nonchildbearing potential. Subjects received a single dose of sotorasib on day 1 and QD doses of itraconazole on days 3 through 7 (with a second [loading]dose of itraconazole on day 3). A single dose of sotorasib was co-administered with itraconazole on day 6. Blood was collected at predetermined timepoints to characterize plasma concentrations of sotorasib and itraconazole. Safety and tolerability monitoring were performed throughout the study.

A total of 14 healthy subjects (2 women and 12 men) were enrolled and completed the study. Nine of the 14 subjects were White and 5 were Black or African American. The mean (SD) age was 44.2 (12.55) years. Single dose plasma sotorasib pharmacokinetic (PK) data were obtained from 14 subjects who received sotorasib alone and sotorasib coadministered with itraconazole following 4 days of multiple daily dosing of itraconazole. In the sotorasib alone and sotorasib with itraconazole treatment periods, sotorasib tₘₐₓ were at 0.51 and 1.00 hour, respectively. Geometric least squares mean ratio (test/reference) of sotorasib AUC_{inf} and Cₘₐₓ were 1.261 and 1.040, respectively, when comparing sotorasib coadministered with itraconazole (test) and sotorasib administered alone (reference) (Table 2).

TABLE 2
Geometric Least Square Means of Sotorasib Pharmacokinetic Parameter
Estimates Following Single Oral Administration of 360 mg Sotorasib
Alone (Day 1) or 360 mg Sotorasib in Combination With 200
mg Itraconazole (Day 6) in Healthy Subjects

| | | | | Test versus |
|---|---|---|---|---|
| | | | | Reference |
| | | | | Ratio of |
| | | | | GLSMs |
| Parameter | Treatment | n | GLSM | (90% CI) |
| AUCₗₐₛₜ | 360 mg Sotorasib | 14 | 22700 | 1.350 |
| (h · ng/mL) | (Reference) | | | (1.141, 1.598) |
| | 360 mg Sotorasib + 200 mg | 13 | 30700 | |
| | Itraconazole (Test) | | | |
| AUC_{inf} | 360 mg Sotorasib | 13 | 24600 | 1.261 |
| (h · ng/mL) | (Reference) | | | (1.119, 1.420) |
| | 360 mg Sotorasib + 200 mg | 13 | 31000 | |
| | Itraconazole (Test) | | | |
| Cₘₐₓ | 360 mg Sotorasib | 14 | 6130 | 1.040 |
| (ng/mL) | (Reference) | | | (0.929, 1.164) |
| | 360 mg Sotorasib + 200 mg | 13 | 6370 | |
| | Itraconazole (Test) | | | |

AUC_{inf} = area under the concentration-time curve from time 0 to infinity;
AUCₗₐₛₜ = area under the concentration time curve from time 0 to the time of the last measurable concentration;
GLSM = geometric least square mean;
CI = confidence interval;
Cₘₐₓ = maximum observed drug concentration;
CV = coefficient of variance;
n = number of subjects with valid observations

### Example 2—an Open-Label Study to Evaluate the Drug-Drug Interaction Effect of Rifampin, a CYP3A4 Inducer, on the Pharmacokinetics of AMG 510 in Healthy Subjects

This was a phase 1, single-center, open-label, fixed-sequence study to investigate the effect of coadministration of rifampin on the PK of sotorasib in healthy men and women. Subjects received a single dose of sotorasib on day 1, a single dose of rifampin (strong OATP1B11/B3 inhibitor) and sotorasib on day 3, QD doses of rifampin on days 5 to 17 and day 19 (strong CYP3A4 inducer). A single dose of sotorasib was co-administered with rifampin on day 18. Blood was collected at predetermined timepoints to characterize plasma concentrations of sotorasib. Safety and tolerability monitoring were performed throughout the study.

A total of 14 healthy subjects (1 woman and 13 men) were enrolled and completed the study. Three of the 14 subjects were White, 10 were Black or African American, and 1 was Asian. The mean (SD) age was 35.2 (8.80) years.

Single dose plasma sotorasib PK data were obtained from 14 subjects who received sotorasib alone (day 1), sotorasib coadministered with a single dose of rifampin (day 3), and sotorasib coadministered with rifampin following 14 days of multiple daily dosing of rifampin (day 18). Median tₘₐₓ were similar across all treatment periods. Geometric mean exposures of sotorasib, based on AUC and Cₘₐₓ, were lower following coadministration with rifampin on days 3 and 18. Geometric least squares mean ratio (test/reference) of sotorasib AUC_{inf} and Cₘₐₓ were 0.766 and 0.840, respectively, when comparing sotorasib coadministered with single-dose rifampin (test) and sotorasib administered alone (reference) (Table 3). Geometric least squares mean ratio (test/reference) of sotorasib AUC_{inf} and Cₘₐₓ were 0.487 and 0.647, respectively, when comparing sotorasib coadministered with multiple daily dosing of rifampin (test) and sotorasib administered alone (reference).

TABLE 3

| | | | | |
|---|---|---|---|---|
| | | | | Test versus |
| | | | | Reference |
| | | | | Ratio of |
| | | | | GLSMs |
| Parameter | Treatment | n | GLSM | (90% CI) |

Day 3

| | | | | |
|---|---|---|---|---|
| AUCₗₐₛₜ | 960 mg Sotorasib on day 1 | 14 | 25400 | 0.765 |
| (h · ng/mL) | (Reference) | | | (0.659, 0.889) |
| | 960 mg Sotorasib + 600 mg | 14 | 19400 | |
| | Rifampin on day 3 (Test) | | | |
| AUC_{inf} | 960 mg Sotorasib on day 1 | 14 | 25600 | 0.766 |
| (h · ng/mL) | (Reference) | | | (0.661, 0.887) |
| | 960 mg Sotorasib + 600 mg | 14 | 19600 | |
| | Rifampin on day 3 (Test) | | | |
| Cₘₐₓ | 960 mg Sotorasib on day 1 | 14 | 6350 | 0.840 |
| (ng/mL) | (Reference) | | | (0.763, 0.925) |
| | 960 mg Sotorasib + 600 mg | 14 | 5340 | |
| | Rifampin on day 3 (Test) | | | |

Day 18

| | | | | |
|---|---|---|---|---|
| AUCₗₐₛₜ | 960 mg Sotorasib on Day 1 | 14 | 25400 | 0.484 |
| (h · ng/mL) | (Reference) | | | (0.417, 0.562) |
| | 960 mg Sotorasib + 600 mg | 14 | 12300 | |
| | Rifampin on Day 18 (Test) | | | |
| AUC_{inf} | 960 mg Sotorasib on Day 1 | 14 | 25600 | 0.487 |
| (h · ng/mL) | (Reference) | | | (0.420, 0.563) |
| | 960 mg Sotorasib + 600 mg | 14 | 12400 | |
| | Rifampin on Day 18 (Test) | | | |
| Cₘₐₓ | 960 mg Sotorasib on Day 1 | 14 | 6350 | 0.647 |
| (ng/mL) | (Reference) | | | (0.588, 0.713) |
| | 960 mg Sotorasib + 600 mg | 14 | 4110 | |
| | Rifampinon Day 18 (Test) | | | |

AUCₗₐₛₜ = area under the concentration-time curve from time 0 to the time of the last measurable concentration;
AUC_{inf} = area under the concentration-time curve from time 0 to infinity;
CI = confidence interval;
Cₘₐₓ = maximum observed drug concentration;
GLSM = geometric least square mean;
n = number of subjects with valid observations

### Example 3—an Open-Label Single-Dose Study to Evaluate the Pharmacokinetics of Sotorasib in Subjects with Moderate or Severe Hepatic Impairment Compared to Healthy Subjects

This Example describes an open-label single-dose study of sotorasib that was conducted in healthy subjects and subjects with moderate or severe hepatic impairment (see, Inclusion/Exclusion criteria):

### Objectives:

The primary objective of the study was to evaluate the pharmacokinetics (PK) of a single oral dose of sotorasib administered in subjects with moderate or severe hepatic impairment compared to subjects with normal hepatic function. The secondary objective of the study was to evaluate the safety and tolerability of sotorasib administered in subjects with moderate or severe hepatic impairment compared to subjects with normal hepatic function.

### Study Design:

This was a Phase 1, parallel-arm, multi-center (US), open-label, non-randomized study to evaluate the PK of a single oral dose of sotorasib administered in subjects with normal hepatic function (control) and in subjects with moderate or severe hepatic impairment (according to Child-Pugh classification) under fasted conditions. On Day 1, after at least a 10-hour fast, all subjects received a single oral dose of 960 mg sotorasib (8×120 mg tablets).

Subjects meeting eligibility requirements were assigned to 1 of 3 groups: Group 1—Normal function (No impairment, n=6-12); Group 2—Moderate impairment (Child-Pugh Class B, n=6-8); Group 3—Severe impairment (Child-Pugh Class C, n=6-8). The classification is based on Child-Pugh scores as per Figg et al. (1995).

Child-Pugh Classification of Severity of Cirrhosis (Table 4)

TABLE 4

| | Points Scored for Each Observed Finding | | |
|---|---|---|---|
| Measurement | 1 | 2 | 3 |
| Encephalopathy¹ | None | Grade 1 or Grade | Grade 3 or Grade |
| | | 2 (or suppressed | 4 (refractory) |
| | | with medication) | |
| Ascites² | Absent | Slight or Subject | Moderate or |
| | | on 1 medication | Severe or Subject |
| | | to control ascites | on 2 medications |
| | | | to control ascites |
| Serum bilirubin (mg/dL) | <2 | 2 to 3 | >3 |
| Serum albumin (g/dL) | >3.5 | 2.8 to 3.5 | <2.8 |
| International | <1.7 | 1.7 to 2.2 | >2.2 |
| Normalized Ratio (INR) | | | |

¹Grade 0: normal consciousness, personality, neurological examination, electroencephalogram
Grade 1: restless, sleep disturbed, irritable/agitated, tremor, impaired handwriting, 5 cycles per second waves
Grade 2: lethargic, time-disoriented, inappropriate, asterixis, ataxia, slow triphasic waves
Grade 3: somnolent, stuporous, place-disoriented, hyperactive reflexes, rigidity, slower waves
Grade 4: unrousable coma, no personality/behavior, decerebrate, slow 2 to 3 cycles per second delta activity
²Ascites is graded according to the following criteria:
Absent: No ascites detectable by manual investigation
Slight: Ascites palpitation doubtful
Moderate: Ascites detectable by palpation
Severe: Necessity of paracentesis, does not respond to medication treatment.

### Inclusion Criteria:

Subjects were required to satisfy all of the following criteria prior to enrollment unless otherwise stated:

### All Subjects

Male subjects or female subjects, between 18 and 70 years of age (inclusive) at the time of Screening.

Body mass index between 18.0 and 38.0 kg/m² (inclusive) at the time of Screening.

Females of nonchildbearing potential defined as permanently sterile (i.e., due to hysterectomy, bilateral salpingectomy, or bilateral oophorectomy) or postmenopausal (defined as at least 45 years of age with amenorrhea for 12 months without an alternative medical cause and follicle-stimulating hormone [FSH]level ?40 mlUlmL).

Subjects with Normal Hepatic Function Only (Group 1)

In good health, determined by no clinically significant findings from medical history, physical examination, 12-lead electrocardiogram (ECG), vital signs measurements, and clinical laboratory evaluations (congenital nonhemolytic hyperbilirubinemia [e.g., suspicion of Gilbert's syndrome based on total and direct bilirubin] is not acceptable) as assessed by the Investigator.

Subiects with Hepatic Impairment Only (Groups 2 and 3)

Child-Pugh B (Group 2) or C (Group 3) classification (see Table 4) defined by both Screening and Check-in clinical laboratory values and clinical examination findings.

Clinically stable hepatic disease in the opinion of the Investigator (e.g., not including rapidly progressive primary or secondary hepatic malignancy). Documented medical history of chronic liver disease including, but not limited to, liver cirrhosis, hepatitis B infection, alcoholic liver disease, or previous hepatitis C virus (HCV) infection (HCV RNA to be undetectable in all enrolled subjects at Screening) or as assessed by the Investigator (or designee).

Subjects with moderate or severe hepatic impairment may have medical findings consistent with their hepatic dysfunction, as determined by medical history, physical examination, 12-lead ECG, vital sign measurements, and clinical laboratory evaluations at Screening and Check-in. Subjects with abnormal findings considered not clinically significant by the Investigator will be eligible.

### Exclusion Criteria:

Subjects were excluded from the study if they satisfied any one of the following criteria prior to enrollment, unless otherwise stated:

### All Subjects

Any unstable medical condition, defined as having been hospitalized within 21 days before Check-in, major surgery within 6 months before Check-in, or otherwise unstable in the judgement of the Investigator and/or Medical Monitor (e.g., risk of complications or adverse events unrelated to study participation).

History or evidence, at Screening or Check-in, of clinically significant disorder, condition, or disease not otherwise excluded that, in the opinion of the Investigator (or designee), would pose a risk to subject safety or interfere with the study evaluation, procedures, or completion.

Venous thromboembolic disease in the last 6 months.

History of malignancy of any type, with the exception of the following: in situ cervical cancer or surgical excised nonmelanomatous skin cancers more than 5 years before receiving sotorasib.

History or evidence of clinically significant arrhythmia at Screening, including any clinically significant findings on the ECG taken at Check-in.

PR interval>200 msec, 2^{nd}-degree atrioventricular (AV) block or 3^{rd}-degree AV block, at Screening or Check-in.

History suggestive of esophageal (including esophageal spasm, esophagitis), gastric, or duodenal ulceration or bowel disease (including but not limited to peptic ulceration, gastrointestinal bleeding, ulcerative colitis, Crohn's disease, or irritable bowel syndrome); or a history of gastrointestinal surgery other than uncomplicated appendectomy cholecystectomy, and hernia repair.

Inability to swallow oral medication or history of malabsorption syndrome.

History of significant hypersensitivity, intolerance, or allergy to any drug compound, food, or other substance, unless approved by the Investigator (or designee) and in consultation with the Sponsor.

Poor peripheral venous access.

Estimated glomerular filtration rate (eGFR) less than 45 mL/min/1.73 m² as calculated by the Modification of Diet in Renal Disease (MDRD) equation, at Screening or Check-in.

Positive human immunodeficiency virus test at Screening.

Use of any over-the-counter or prescription medications within 30 days or 5 half-lives (whichever is longer) before enrollment, with below exceptions:

Ibuprofen and hormone-replacement therapy (e.g., estrogen, thyroid) will be allowed.
Therapies for hepatic disease and treatments of associated disorders that have been stable for at least 30 days prior to study drug administration and deemed acceptable, by the Investigator (or designee) and the Sponsor, to be given concurrently with sotorasib during the study period.

Administration of an approved (authorized) Coronavirus Disease 2019 (COVID-19) vaccine in the past 28 days prior to dosing or a COVID-19 vaccine given Emergency Use Approvals (US) in the past 30 days prior to dosing.

All herbal medicines (e.g., St. John's wort), vitamins, and supplements consumed by the subject within the 30 days prior to enrollment, unless deemed acceptable by the Investigator (or designee) and in consultation with the Sponsor.

Consumption of foods and beverages containing poppy seeds, grapefruit, or Seville oranges within 7 days prior to Check-in.

Use of known CYP3A4 and P-gp-sensitive substrates (with a narrow therapeutic window), within 30 days or 5 half-lives of the drug or its major active metabolite, whichever is longer, prior to study Day 1 that was not reviewed and approved by the Investigator (or designee) and the Sponsor.

Use of strong inducers of CYP3A4 (including herbal supplements such as St. John's wort) within 30 days or 5 half-lives (whichever is longer) prior to study Day 1.

Use of PPIs within 5 days or H2-receptor antagonists within 1 day prior to study Day 1.

History of alcoholism or drug/chemical abuse within the last 3 months prior to Check-in.

Alcohol consumption from 48 hours prior to Check-in.

Regular alcohol consumption of >14 units per week for males and >7 units for females. One unit of alcohol equals 12 oz (360 mL) beer, 1%/2 oz (45 mL) liquor, or 5 oz (150 mL) wine.

Use of tobacco- or nicotine-containing products within 3 months prior to Check-in.

Positive test for illicit drugs, cotinine (tobacco or nicotine use), and/or alcohol use at Screening or Check-in.

Consumption of caffeine-containing foods and beverages within 48 hours prior to Check-in.

Female subjects with a positive pregnancy test at Screening or Check-in.

Male subjects (with a female partner of childbearing potential) who are unwilling to practice sexual abstinence (refrain from heterosexual intercourse) or unwilling to adhere to contraceptive requirements through 7 days after sotorasib dosing.

Unwilling to abstain from sperm donation and ovum donation through 7 days after sotorasib dosing.

Male subjects with a female partner of childbearing potential and not willing to inform his partner of his participation in this clinical study.

Male subjects with a pregnant partner or partner planning to become pregnant who are unwilling to practice abstinence or use a condom for 7 days after sotorasib dosing.

Subject has received a dose of an investigational drug (new chemical entity) within the past 30 days or 5 half-lives, whichever is longer, prior to Check-in.

Have previously completed or withdrawn from this study or any other study investigating sotorasib or have previously received the investigational product.

Donation of blood from 3 months prior to Check-in, plasma from 2 weeks prior to Check-in, or platelets from 6 weeks prior to Check-in.

Receipt of blood products within 2 months prior to Check-in.

Unwilling to abide with study restrictions.

Subjects who, in the opinion of the Investigator (or designee), should not participate in this study.

Subjects with Normal Hepatic Function Only (Group 1)

Positive hepatitis B or hepatitis C panel at Screening. Subjects whose results are compatible with prior immunity (vaccination or prior infection) may be included.

Alanine aminotransferase (ALT) or aspartate aminotransferase (AST)>upper limit of normal (ULN) at Screening or Check-in.

Total bilirubin levels>ULN at Screening or Check-in.

A QT interval corrected for heart rate based on the Fridericia correction (QTcF) interval>450 msec in male subjects or >470 msec in female subjects or history/evidence of long QT syndrome at Screening or Check-in, confirmed by calculating the mean of the original value and 2 repeats.

Subjects with Hepatic Impairment Only (Groups 2 and 3)

Values outside the normal range for liver function tests that are not consistent with their hepatic condition, as determined by the Investigator (or designee).

A QTcF interval>470 msec in male subjects or >480 msec in female subjects at Screening or Check-in, confirmed by calculating the mean of the original value and 2 repeats.

Use of a new medication, or a change in dose, for the treatment, or worsening of, hepatic encephalopathy within 30 days prior to Check-in.

Recent history of, or the treatment of, gastrointestinal bleeding (within the past 6 months).

Presence of a portosystemic shunt.

Recent history of paracentesis within 30 days prior to Check-in.

Current functioning organ transplant or are waiting for an organ transplant.

Evidence of severe ascites.

History within 60 days prior to the Screening visit or current symptoms of hepatic encephalopathy Grade 2 or above.

### Concomitant Therapies:

Subjects will refrain from use of any prescription or nonprescription medications/products during the study until the EOS, unless the Investigator (or designee) and/or Sponsor have given their prior consent.

Subjects will refrain from use of any PPIs within 5 days or H2-receptor antagonists within 1 day prior to study Day 1 until at least 4 hours after IMP administration.

For hepatically impaired subjects, treatment for the underlying liver disease and comorbid conditions (including prescribed analgesia) will be permitted if prescribed by the subject's personal physician and approved by the Medical Monitor and Investigator, in consultation with the Sponsor as needed. Administration of medications should be withheld for at least 4 hours after study drug administration as clinically appropriate, unless needed for treatment of an adverse event, at the discretion of the Investigator. Throughout the study, the Investigator may prescribe any concomitant medications or treatments deemed necessary to provide adequate supportive care except for those listed in the exclusion criteria.

Foods and medicines that are known strong CYP3A4 inducers (e.g., rifampin, corticosteroids, anticonvulsants, and St. John's wort) or CYP3A4 or P-gp substrates with narrow therapeutic index are prohibited prior to IMP administration as specified in the exclusion criteria above and during the study until the end of study.

Ibuprofen and hormone-replacement therapy are acceptable concomitant medications. The administration of any other concomitant medications during the study is prohibited without prior approval of the Investigator (or designee), unless its use is deemed necessary for treatment of an adverse event. Any medication taken by a subject during the course of the study and the reason for its use will be documented in the source data.

### Pharmacokinetic Analyses:

The plasma PK parameters of sotorasib will be calculated using standard noncompartmental methods.

The primary PK parameters are Cₘₐₓ, AUCₗₐₛₜ, and AUC_{inf} for sotorasib. Other PK parameters for sotorasib will not be subject to inferential statistical analysis and may include time of tₘₐₓ, apparent plasma terminal elimination half-life (t_{1/2,z}), apparent total plasma clearance (CL/F), apparent volume distribution during the terminal elimination phase (V_{z}/F), unbound fraction (fᵤ), C_{max,u}, AUC_{last,u}, AUC_{inf,u}, CLᵤ/F, and V_{z,u}/F. A linear model will be used to analyze log-transformed primary PK parameters. The data from hepatically impaired subjects (Group 2 and 3; tests) and control subjects (normal hepatic function [Group 1]; reference) will be included in the analysis. Geometric mean ratios (Test/Reference) for Cₘₐₓ and AUC values and associated 90% confidence intervals will be estimated.

Additional parameters may be calculated, and sotorasib metabolites may be analyzed. PK parameters for the metabolites, if calculated, will not be subject to inferential statistical analysis. Specific details will be presented in the Statistical Analysis Plan for this study.

### Study Results:

### Pharmacokinetic Results:

Following single dose administration of 960 mg sotorasib to subjects with normal hepatic function and subjects with moderate or severe hepatic impairment, median tₘₐₓ values were similar, ranging between 1.00 and 1.47 hours. Estimated half-lives were similar across the groups (arithmetic mean range of 6.42 to 8.18 hours), with no apparent trend related to hepatic impairment.

The geometric least squares mean (GLSM) ratios for test/reference (90% Cl) for subjects with moderate hepatic impairment compared to subjects with normal hepatic function were 0.746 (0.431, 1.29), 0.749 (0.431, 1.30), and 0.955 (0.512, 1.78) for AUC_{inf}, AUCₗₐₛₜ, and Cₘₐₓ, respectively. The GLSM ratios for test/reference (90% Cl) for subjects with severe hepatic impairment compared to subjects with normal hepatic function were 1.04 (0.545, 1.97), 1.04 (0.544, 1.99), and 1.43 (0.688, 2.96) AUC_{inf}, AUCₗₐₛₜ, and Cₘₐₓ, respectively.

Metabolite M10 appeared in plasma with the median tₘₐₓ occurring between 6 and 8 hours postdose, which was approximately 2 hours faster in the subjects in moderate and severe hepatic impairment group when compared with subjects with normal hepatic function. The geometric mean Cₘₐₓ and AUC values increased with increasing severity of hepatic impairment such that geometric mean exposure in hepatically impaired subjects was higher than that of normal subjects.

For all groups, metabolite M18 median tₘₐₓ occurred between 2 and 2.55 hours postdose. The geometric mean Cₘₐₓ and AUC values decreased with increasing severity of hepatic impairment. Metabolite M18 exposure was highest in normal subjects. Arithmetic mean t_{1/2} values for M18 were similar for subjects with normal hepatic function and subjects with moderate and severe hepatic impairment, with values between 7.28 and 9.35 hours.

Metabolite M24 appeared in plasma with the median tₘₐₓ occurring between 4.00 and 7.97 hours postdose, with high variability noted in all groups. The geometric mean Cₘₐₓ and AUC values increased with increasing severity of hepatic impairment, with mean AUC_{inf} higher for severely impaired subjects. Arithmetic mean t_{1/2} values for M24 was similar for subjects with normal hepatic function and subjects with moderate and severe hepatic impairment, with values between 23.0 and 28.2 hours.

### Safety Results:

Of the 20 subjects, 4 subjects (20%) reported 9 treatment-emergent adverse events during the study. Of these 5 treatment-emergent adverse events were considered by the Investigator to be related to sotorasib.

All the adverse events were considered mild in severity. There were no serious adverse events or treatment-emergent adverse events leading to discontinuation from the study. All the adverse events resolved by the end of study. There were no clinically meaningful findings in clinical laboratory evaluations, physical examinations, ECGs, and vital signs measurements during the study.

### Conclusions:

Sotorasib AUC_{inf}, AUCₗₐₛₜ, and Cₘₐₓ GLSM ratios for test/reference (90% CI) for subjects with moderate hepatic impairment compared to subjects with normal hepatic functions were 0.746 (0.431, 1.29), 0.749 (0.431, 1.30), and 0.955 (0.512, 1.78), respectively.

Sotorasib AUC_{inf}, AUCₗₐₛₜ, and Cₘₐₓ GLSM ratios for test/reference (90% CI) for subjects with severe hepatic impairment compared to subjects with normal hepatic functions were 1.04 (0.545, 1.97), 1.04 (0.544, 1.99) and 1.43 (0.688, 2.96), respectively.

Arithmetic mean t_{1/2} values for sotorasib were similar in subjects with normal hepatic function and subjects with moderate or severe hepatic impairment.

Exposures (based on Cₘₐₓ and AUC values) to metabolites M10 and M24 increased with increasing severity of hepatic impairment whereas the exposure to metabolite M18 decreased with increasing severity of hepatic impairment.

Single doses of sotorasib were safe and well-tolerated when administered to healthy subjects with normal hepatic function and subjects with moderate of severe hepatic impairment.

### REFERENCES

Albert et al., (2007) Nat. Methods, 4:903-905.
Beers and Nederlof, (2006) Breast Cancer Res., 8(3):210.
Bertone et al., (2006) Genome Res., 16(2):271-281.
Canon et al., (2019) Nature, 575:217-223.
Cerami et al., Cancer Discov. 2012, 2(5), 401.
Chung et al., (2004) Genome Res., 14(1):188-196.
Cully and Downward, (2008) CELL, 133:1292.
Dalma-Weiszhausz et al., (2006) Methods Enzymol., 410:3-28.
DeRisi et al., (1996) Nat. Genet., 14:457-460.
Figg et al., (1995) Pharmacotherapy, 15:693-700.
Forshew et al., (2012) Sci Transl Med., 4:136ra68
Gao et al., Science Signaling 2013, 6(269), pl1.

Guidance for the Industry, Pharmacokinetics in Patients with Impaired Hepatic Function: Study Design, Data Analysis and Impact on Dosing and Labeling, U.S. Department of Health and Human Services, Food and Drug Administration, Center for Drug Evaluation and Research (CDER), Center for Biologics Evaluation and Research (CBER), May 2003 (available at: https://www.fda.gov/regulatory-information/search-fda-guidance-documents/pharmacokinetics-patients-impaired-hepatic-function-study-design-data-analysis-and-impact-dosing-and, last accessed Oct. 14, 2022).
Haber and Velculescu, (2014) Cancer Discov., 4:650-661.
Hong et al., (2020), N. Engl. J. Med., 383, 1207.
Hughes et al., (2001) Nat. Biotechnol., 19(4):342-347.
Irizarry, (2003) Nucleic Acids Res., 31:e15.
Janes et al., (2018) Cell, 172(3):578-589.
Jasmine et al., (2012) PLoS One, 7(2):e31968.
Kim et al., (2006) Carcinogenesis, 27(3):392-404.
Kinde et al., (2011) Proc Natl Acad Sci USA, 108:9530-5.
Kumar et al., (2012) J. Pharm. Bioallied Sci., 4(1):21-26.
Laere et al., (2009) Methods Mol. Biol., 512:71-98.
Lanman et al., (2020) J. Med. Chem., 63:52.
Lin et al., (2010) BMC Genomics, 11:712.
Liu et al., (2017) Biosens Bioelectron., 92:596-601.
Lodes et al., (2009) PLoS One, 4(7):e6229.
LUMAKRAS® US Prescribing Information, Amgen Inc., Thousand Oaks, California, 91320 (revision May 2021)
Mackay et al., (2003) Oncogene, 22:2680-2688.
Mao et al., (2007) Curr. Genomics, 8(4):219-228.
McDonald et al., (2017) Cell, 170(3):577-592.
Michels et al., (2007) Genet. Med., 9:574-584.
Mockler and Ecker, (2005) Genomics 85(1):1-15.
Ostrem et al., (2013) Nature, 503:548-551.
Ostrem and Shokat, (2016) Nature Rev. Drug Discov., 15(11):771-785.
Patricelli et al., (2016) Cancer Discovery, 6:316-329.
Pinkel et al., (2005) Nat. Genetics, 37:S11-S17.
Simanshu et al., (2017) CELL, 170:17-33.
Thomas et al., (2005) Genome Res., 15(12):1831-1837.
Thompson et al., (2012) PLoS ONE, 7:e31597.
Wang et al., (2012) Cancer Genet., 205(7-8):341-55.
Wei et al., Nucleic Acids Res 2008; 36(9):2926-2938.
Xie et al., (2017) Front Pharmacol., 8:823.

What is claimed is:

1. A method of treating cancer in a patient comprising administering a therapeutically effective amount of sotorasib to the patient, wherein the patient had moderate or severe hepatic impairment prior to administration of the sotorasib, wherein sotorasib is a compound of Formula (I)

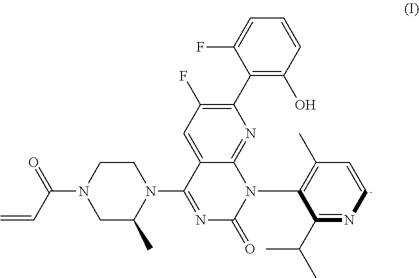

2. The method of claim 1, wherein the patient had moderate hepatic impairment prior to administration of the sotorasib.

3. The method of claim 1, wherein the patient had severe hepatic impairment prior to administration of the sotorasib.

4. The method of claim 1 or claim 2, wherein the method further comprises a step of determining whether the patient has moderate hepatic impairment.

5. The method of claim 1 or claim 3, wherein the method further comprises a step of determining whether the patient has severe hepatic impairment.

6. The method of any one of claims 1 to 5, wherein the therapeutically effective amount of sotorasib is 240 mg to 960 mg daily.

7. The method of any one of claims 1 to 6, wherein the therapeutically effective amount of sotorasib is 240 mg sotorasib daily.

8. The method of any one of claims 1 to 6, wherein the therapeutically effective amount of sotorasib is 480 mg daily.

9. The method of any one of claims 1 to 6, wherein the therapeutically effective amount of sotorasib is 960 mg daily.

10. The method of any one of claims 1 to 9, wherein the patient has a cancer comprising a KRAS G12C mutation.

11. The method of claim 10, wherein the method further comprises determining whether the patient has a cancer comprising a KRAS G12C mutation.

12. The method of any one of claims 1 to 11, wherein the cancer is a solid tumor.

13. The method of any one of claims 1 to 11, wherein the cancer is non-small cell lung cancer, small bowel cancer, appendiceal cancer, colorectal cancer, cancer of unknown primary, endometrial cancer, pancreatic cancer, hepatobiliary cancer, small cell lung cancer, cervical cancer, germ cell cancer, ovarian cancer, gastrointestinal neuroendocrine cancer, bladder cancer, myelodysplastic/myeloproliferative neoplasms, head and neck cancer, esophagogastric cancer, soft tissue sarcoma, mesothelioma, thyroid cancer, leukemia, melanoma, ampullary cancer, gastric cancer, sinonasal cancer, or bile duct cancer.

14. The method of any one of claims 1 to 11, wherein the cancer is non-small cell lung cancer, small bowel cancer, appendiceal cancer, colorectal cancer, cancer of unknown primary, endometrial cancer, pancreatic cancer, melanoma, ampullary cancer, gastric cancer, sinonasal cancer, or bile duct cancer.

15. The method of any one of claims 1 to 11, wherein the cancer is non-small cell lung cancer.

16. The method of claim 15, wherein the non-small cell lung cancer is locally advanced or metastatic.

17. The method of any one of claims 1 to 11, wherein the cancer is colorectal cancer.

18. The method of any one of claims 1 to 11, wherein the cancer is pancreatic cancer.

19. The method of any one of claims 1 to 18, where the sotorasib is administered as one or more tablets.

20. The method of any one of claims 1 to 19, wherein the sotorasib is administered orally.

21. The method of any one of claims 1 to 20, wherein the sotorasib is administered once daily.
